(19) United States
(12) Patent Application Publication
(10) Pub. No.: US 2016/0264924 A1
(43) Pub. Date: Sep. 15, 2016

(54) Methods And Apparatuses For Droplet Mixing

(71) Applicant: GigaGen, Inc., San Francisco, CA (US)

(72) Inventors: David Scott Johnson, San Francisco, CA (US); Gary Withey, San Francisco, CA (US); Philip Homewood, Middlesex (GB); Timothy Atkins, Middlesex (GB)

(21) Appl. No.: 15/160,671

(22) Filed: May 20, 2016

Related U.S. Application Data
(62) Division of application No. 14/368,091, filed on Jun. 23, 2014.
(60) Provisional application No. 61/580,105, filed on Dec. 23, 2011.

(51) Int. Cl. C12N 1/06; B01L 3/00

(52) U.S. Cl. CPC C12N 1/06; B01L 3/502715; B01L 3/502784; B01L 3/50273; B01L 2300/0867; B01L 2200/0647; B01L 2300/18

(57) ABSTRACT

Methods and systems are provided for merging a droplet with a volume of fluid in a microfluidic system. In particular, the methods of the invention use a microfluidic structure designed to merge a fluid with a droplet in order to dilute, add volume, or add selected reagents, biological materials, or synthetic materials to a droplet. Also provided are related systems and methods for cell lysis.

### CROSS-REFERENCE TO RELATED APPLICATIONS

This application is a divisional of U.S. application Ser. No. 14/368,091, filed Jun. 23, 2014, which claims benefit of PCT/US2012/070989, filed Dec. 20, 2012, which claims the benefit of U.S. Provisional Patent Application No. 61/580,105, filed Dec. 23, 2011, the disclosures of which are each incorporated herein by reference in their entirety.

### FIELD OF THE INVENTION

The invention relates to apparatuses and methods for merging a volume of fluid with a droplet in a microfluidic device. In particular, the methods of the invention use a microfluidic structure designed to merge a fluid with a droplet in order to dilute, add volume, or add selected reagents, biological materials, or synthetic materials to a droplet.

### BACKGROUND OF THE INVENTION

There has been a growing interest in the application of microfluidic systems to a variety of technical areas, including such diverse fields as biochemical analysis, medical diagnostics, chemical synthesis, and environmental monitoring. Microfluidic systems provide certain advantages in acquiring chemical and biological information. For example, microfluidic systems permit complicated processes to be carried out using very small volumes of fluid, thus minimizing consumption of both samples and reagents. Chemical and biological reactions occur more rapidly when conducted in microfluidic volumes. Furthermore, microfluidic systems permit large numbers of complicated biochemical reactions and/or processes to be carried out in a small area (such as within a single integrated device) and facilitate the use of common control components. Examples of desirable applications for microfluidic technology include processes, such as analytical chemistry; chemical and biological synthesis; DNA amplification; and screening of chemical and biological agents for activity.

Microdroplets can be used to perform a variety of biochemical and molecular biological tasks. Such microdroplets are advantageous due to their small (i.e., picoliter) scale, their stability, and their ability to be processed in a high throughput method, such as in a microfluidic device. However, certain methods of analysis require addition of reagents or fluid to a droplet after droplet formation, which is a challenge. What is needed, therefore, is a new system and method for droplet merging or dilution in a microfluidic device.

### SUMMARY OF THE INVENTION

The present disclosure addresses long-felt needs in the field of microfluidic devices by providing apparatuses and methods for merging or diluting droplets in a microfluidic channel. In one embodiment, a microfluidic device comprises a first microchannel defining a first fluid path, wherein the first microchannel comprises an inlet portion comprising an inlet portion cross-sectional area and a first flow axis. The first microchannel also comprises a chamber portion in fluid communication with and adjacent to the inlet portion, comprising a maximal cross-sectional area and a second flow axis. The chamber portion comprises a fluid junction comprising a fluid junction cross-sectional area and a constriction comprising a constriction cross-sectional area. The constriction cross-sectional area is less than the maximal cross-sectional area of the chamber portion. The first microchannel also comprises an outlet portion in fluid communication with and adjacent to the chamber portion, comprising an outlet portion cross-sectional area and a third flow axis, wherein the outlet portion cross-sectional area is greater than or equal to the constriction cross-sectional area. This embodiment further comprises a second microchannel defining a second fluid path and a fourth flow axis, where the second fluid path terminates at one end at the fluid junction of the first microchannel.

In one embodiment, the constriction cross-sectional area is less than the sum of the inlet portion cross-sectional area and the fluid junction cross-sectional area. In another embodiment, the maximal cross-sectional area is at least four times greater than the fluid junction cross-sectional area. In yet another embodiment, the maximal cross-sectional area is at least two times greater than the fluid junction cross-sectional area. In still another embodiment, the maximal cross-sectional area is at least nine times greater than the inlet portion cross-sectional area.

In one aspect, the first flow axis intersects the fourth flow axis at an angle of 0 to 180 degrees. In another aspect, the first flow axis intersects the fourth flow axis at an angle of zero to ninety degrees. In still another aspect, the first flow axis is orthogonal to the fourth flow axis. In yet another aspect, the first flow axis intersects the second flow axis at an angle of zero to 90 degrees.

Also provided are methods for merging, diluting, or increasing the volume of droplets, comprising flowing a first solution comprising an initial droplet in an immiscible carrier liquid through a first microchannel defining a first fluid path from an inlet portion into a chamber portion. The chamber portion comprises a constriction downstream from the inlet portion. The methods also include flowing a second solution from a second microchannel through a fluid junction to the chamber portion, wherein the second solution is miscible with the initial droplet, and the initial droplet and the second solution merge within the chamber portion, thereby increasing the volume of the initial droplet and forming a merged droplet. In one embodiment, this method is used, for example, to dilute the contents of a droplet, or to add one or more reagents, biological materials, or synthetic materials to a droplet. In a further embodiment, the reagents, biological materials, or synthetic materials are provided to the droplet to perform a biochemical analysis on the contents of the droplet.

In one aspect, a first solution flows into the chamber from the inlet portion, a second solution flows into the chamber portion from the second microchannel, and the merged droplet formed in the chamber portion flows out through the constriction and into the outlet portion. In a further aspect, the constriction has a cross-sectional area sufficient to produce a pressure gradient within the chamber portion. In one embodiment, the merged droplet flows through the constriction and the flow of the first and second solutions through the constriction produces a pressure gradient within the chamber portion. In another embodiment, a high pressure zone is created near the constriction, helping to induce the merger of the droplet and the second solution.

In one aspect, the initial droplet diameter is less than 300 μm. In another aspect, the rate of formation of the merged droplets is greater than 1, 5, 10, 20, 50, 75, or 100 Hz. In still another aspect, the rate of formation of the merged droplets is 100 Hz. In yet another aspect, the first solution or the second solution flows under pressure generated by a pump.

In one embodiment, the first solution and the second solution are polar. In another embodiment, the first solution and the second solution are non-polar.

In one aspect, the second solution comprises a reagent, biological material, or synthetic material to be added to the initial droplet. In another aspect, the second solution dilutes the initial droplet.

In one embodiment, the initial droplet comprises a cell. In a further embodiment, the second solution is hypotonic as compared to tonicity of the initial droplet. In another further embodiment, the hypotonicity of the second solution promotes lysis of the cell. In another further embodiment, chemicals in the second solution promote lysis of the cell. In certain such embodiments, chemical lysis is induced by a surfactant such as Triton X-100, Tween 20, or NP40. In other such embodiments, chemical lysis occurs with an enzyme such as proteinase K. In still another embodiment, chemical lysis occurs at room temperature. In yet another embodiment, chemical lysis occurs at 90-98° C.

Also provided in the present disclosure is a method for cell lysis, comprising flowing a first solution comprising a cell encapsulated in an initial droplet in an immiscible carrier liquid through a first microchannel defining a first fluid path from an inlet portion into a chamber portion. The chamber portion comprises a constriction downstream from the inlet portion. The method also comprises flowing a second solution from a second microchannel through a fluid junction to the chamber portion, wherein the second solution comprises a cell lysis solution miscible with the initial isotonic droplet. The initial isotonic droplet and the second solution merge within the chamber portion, thereby diluting the initial isotonic droplet with the second solution, creating a merged droplet. The cell encapsulated in the merged droplet lyses.

In one embodiment, the first solution is isotonic with respect to said cell. In another embodiment, the cell lysis solution is hypotonic with respect to the initial droplet solution. In yet another embodiment, the cell lysis solution is hypotinic with respect to said cell. In a further embodiment, the hypotonic solution comprises reagents, biological materials, or synthetic materials for biochemical analysis. In another aspect, the cell lysis solution comprises a chemical that promotes lysis of the cell. In a further aspect, the chemical is a surfactant. In some aspects, the surfactant is Triton X-100, Tween 20, or NP 40. In other aspects, the chemical is an enzyme, i.e., proteinase K. In some embodiments, cell lysis occurs at room temperature. In other embodiments, lysis of the cell occurs at 90-98° C.

In one embodiment, the method further comprises biochemical analysis of components of said lysed cell in said merged droplet after cell lysis. In a further embodiment, the biochemical analysis is nucleic acid amplification. In another embodiment, the cell lysis solution is not purified or treated prior to biochemical analysis.

In one aspect, the initial droplet comprises a concentration of salt, protein, or other chemical. In a further aspect, this concentration enhances the stability of the cell. In still another aspect, the cell lysis solution has at least one order of magnitude lower concentration of salt, protein, or other chemical than the first solution. In yet another aspect, the initial droplet has less than one microliter of isotonic buffer.

In one embodiment, the initial droplet comprises a plurality of cells. In another embodiment, at least one of the plurality of cells is lysed after the initial droplet merges with the second solution.

Also provided in the present disclosure is a method for cell lysis, comprising flowing a first solution comprising a cell encapsulated in an initial isotonic droplet in an immiscible carrier liquid through a first microchannel defining a first fluid path from an inlet portion into a chamber portion. The chamber portion comprises a constriction downstream from the inlet portion. The method also comprises flowing a second solution from a second microchannel through a fluid junction to the chamber portion, wherein the second solution comprises a chemical solution miscible with the initial droplet. The initial droplet and the second solution merge within the chamber portion, thereby adding chemicals for cell lysis at the appropriate concentration. The cell encapsulated in the merged droplet then lyses.

### BRIEF DESCRIPTION OF THE FIGURES

FIG. 1 is a schematic view of a microchannel configuration for droplet merging.

FIG. 2, A-M are schematic views of selected embodiments of microchannel designs and configurations.

FIG. 3 is a schematic view of a microchannel configuration for droplet merging, with preferred ranges for aspects of the microfluidic channel designs.

FIG. 4, panels A-D depict steps in a single droplet merging in one aspect of a microfluidic device of the invention.

FIG. 5 is a schematic of a microfluidic method for cell lysis using dilution with a hypotonic buffer.

FIG. 6 is a schematic of a microfluidic chip for cell lysis and subsequent addition of amplification reagents.

FIG. 7 is a flowchart depicting the steps for droplet merging in a microfluidic device.

FIG. 8 is a flowchart depicting steps for cell lysis in a microfluidic device.

### DETAILED DESCRIPTION OF THE INVENTION

The following terms, unless otherwise indicated, shall be understood to have the following meanings:

“About,” as used herein, when referring to a measurable value, such as an amount, a temporal duration, and the like, is meant to encompass variations of ±20% or ±10%, more preferably ±5%, even more preferably ±1%, and still more preferably ±0.1% from the specified value, as such variations are appropriate to perform the disclosed methods.

Throughout this specification and claims, the word “comprise” or variations such as “comprises” or “comprising,” will be understood to imply the inclusion of a stated integer or group of integers but not the exclusion of any other integer or group of integers.

The term “microfluidic,” as used herein, refers to structures or devices through which one or more fluids are capable of being passed or directed and that have at least one dimension less than about 500 microns.

The terms “emulsion droplet” or “emulsion microdroplet” refer to a droplet that is formed when two immiscible fluids are combined. For example, an aqueous droplet can be formed when an aqueous fluid is added to a non-aqueous fluid. In another example, a non-aqueous fluid can be added to an aqueous fluid to form a droplet. The droplets of an emulsion may have any uniform or non-uniform distribution. Any of the emulsions disclosed herein may be monodisperse, that is, composed of droplets of at least generally uniform size, or may be polydisperse, that is, composed of droplets of various sizes. If monodisperse, the droplets of the emulsion may vary in volume by a standard deviation that is less than about plus or minus 100%, 50%, 20%, 10%, 5%, 2%, or 1% of the average droplet volume. Droplets generated from an orifice may be monodisperse or polydisperse.

An emulsion may have any suitable composition. The emulsion may be characterized by the predominant liquid compound or type of liquid compound that is used. The predominant liquid compounds in the emulsion may be water and oil. “Oil” is any liquid compound or mixture of liquid compounds that is immiscible with water and that has a high content of carbon. In some examples, oil also may have a high content of hydrogen, fluorine, silicon, oxygen, or any combination thereof, among others. For example, any of the emulsions disclosed herein may be a water-in-oil (W/O) emulsion (i.e., aqueous droplets in a continuous oil phase). The oil may be or include at least one silicone oil, mineral oil, fluorocarbon oil, vegetable oil, or a combination thereof, among others. Any other suitable components may be present in any of the emulsion phases, such as at least one surfactant, reagent, biological material, synthetic material, sample (i.e., partitions thereof), other additive, label, particles, or any combination thereof.

“Droplet” refers to a small volume of liquid, typically with a spherical shape, encapsulated by an immiscible fluid. The volume of a droplet, and/or the average volume of droplets in an emulsion, may, for example, be less than about one microliter (i.e., a “microdroplet”) (or between about one microliter and one nanoliter, or between about one microliter and one picoliter), less than about one nanoliter (or between about one nanoliter and one picoliter), or less than about one picoliter (or between about one picoliter and one femtoliter), among others. A droplet (or droplets of an emulsion) may have a diameter (or an average diameter) of less than about 1000, 100, or 10 micrometers, or of about 1000 to 10 micrometers, among others. A droplet may be spherical or nonspherical. In some embodiments, the droplet has a volume and diameter that is large enough to encapsulate a cell.

The term “reagent” as used herein refers to any chemical compound used alone or in combination with a different chemical compound to produce a desired chemical reaction. The term “reagent” includes all catalysts (transition metal based or otherwise), ligands, acids, bases and other materials that are added to a reaction mixture in order to provide the desired result.

As used herein, the term “biological material” is intended to mean any substance derived or obtained from a living organism. Illustrative examples of biological materials include, but are not limited to, the following: cells; tissues; cell or tissue fragments; blood or blood components; proteins, including recombinant and transgenic proteins, and proteinaceous materials; enzymes, including digestive enzymes, such as trypsin, chymotrypsin, alpha-glucosidase and iduronodate-2-sulfatase; immunoglobulins, including mono and polyimmunoglobulins; botanicals; food and the like.

The term “synthetic material” refers to compounds or materials formed through a chemical process by a human agency, as opposed to those of natural origin. Illustrative examples of synthetic materials include, but are not limited to, the following: microparticles, nanoparticles, nanowires, microbeads, and the like.

In general, microfluidic devices are planar in structure and are constructed from an aggregation of planar substrate layers, wherein the fluidic elements, such as microchannels, etc., are defined by the interface of the various substrate layers. The microchannels, etc. are usually etched, embossed, molded, ablated, or otherwise fabricated into a surface of a first substrate layer as grooves, depressions, or the like. A second substrate layer is subsequently overlaid on the first substrate layer and bonded to it in order to cover the grooves, etc., in the first layer, thus creating sealed fluidic components within the interior portion of the device. Optionally, either one or both substrate layers have microchannels devised within them. Such microchannels can be aligned one on top of another when the substrate layers are joined together. Such microchannels as thus constructed can be symmetrical (i.e., the microchannel on the first substrate is the same shape as that of the microchannel on the second substrate, thus forming a symmetrical microchannel when the two substrate layers are joined) or such microchannels can be asymmetrical (i.e., the microchannel on the first substrate is a different shape from that of the microchannel on the second substrate, thus forming an asymmetrical channel when the two substrate layers are joined). Additionally, open-well elements can be formed by making perforations in one or more substrate layers, which perforations optionally can correspond to depressed microreservoir, microchannel, etc., areas on the complementary layer. This paragraph provides one example of how microfluidic devices may be generally constructed, but a variety of other designs may be used or modifications made to this design.

Manufacturing of these microscale elements into the surface of the substrates can be carried out through any number of microfabrication techniques that are well known in the art. For example, lithographic techniques are optionally employed in fabricating, e.g., glass, quartz, or silicon substrates, using methods well known in the semiconductor manufacturing industries, such as photolithographic etching, plasma etching, or wet chemical etching. Alternatively, micromachining methods, such as laser drilling, micromilling, and the like are optionally employed. Similarly, for polymeric substrates, well known manufacturing techniques may also be used. These techniques include injection molding or stamp molding methods, wherein large numbers of substrates are optionally produced using, e.g., rolling stamps to produce large sheets of microscale substrates, or polymer microcasting techniques where the substrate is polymerized within a micromachined mold. Furthermore, various combinations of such techniques are optionally combined to produce the microelements present in the current invention.

As stated above, the substrates used to construct the microfluidic devices of the invention are typically fabricated from any number of different materials, depending upon, e.g., the nature of the samples to be assayed, the specific reactions and/or interactions being assayed for, etc. For some applications, the substrate can optionally comprise a solid non-porous material. For example, the substrate layers can be composed of, e.g., silica-based materials (such as glass, quartz, silicon, fused silica, or the like), polymeric materials or polymer coatings on materials (such as polymethylmethacrylate, polycarbonate, polytetrafluoroethylene, polyvinylchloride, polydimethylsiloxane, polysulfone, polystyrene, polymethylpentene, polypropylene, polyethylene, polyvinylidine fluoride, acrylonitrile-butadiene-styrene copolymer, parylene or the like), ceramic materials, metal materials, etc.

The surface of a substrate layer may be of the same material as the non-surface areas of the substrate or, alternatively, the surface may comprise a coating on the substrate base. Furthermore, if the surface is coated, the coating optionally can cover either the entire substrate base or can cover select subparts of the substrate base. For example, in the case of glass substrates, the surface of the glass of the base substrate may be treated to provide surface properties that are compatible and/or beneficial to one or more samples, reagents, biological materials, or synthetic materials being used. Such treatments include derivatization of the glass surface, e.g., through silanization or the like, or through coating of the surface using, e.g., a thin layer of other material such as a polymeric or metallic material. Derivatization using silane chemistry is well known to those of skill in the art and can be readily employed to add, e.g., amine, aldehyde, or other functional groups to the surface of the glass substrate, depending upon the desired surface properties. Further, in the case of metal substrates, metals that are not easily corroded under potentially high salt conditions, applied electric fields, and the like are optionally preferred.

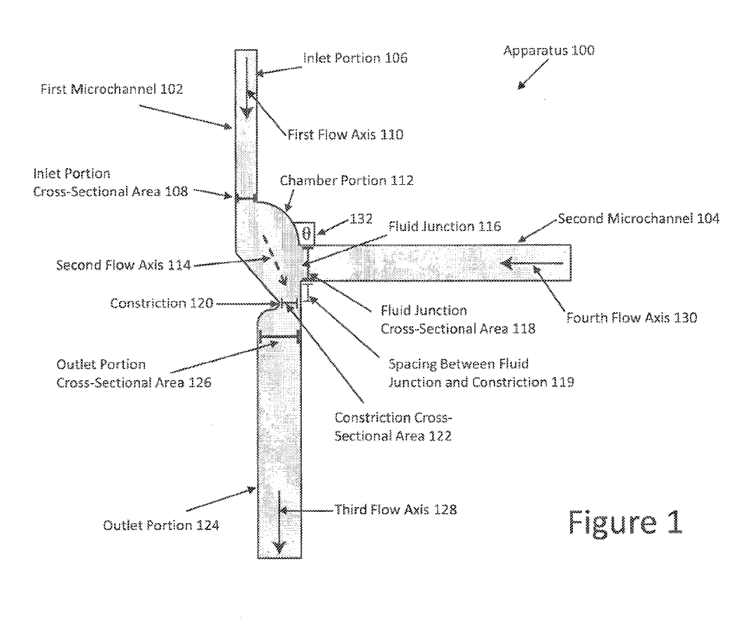

Although described in terms of a layered planar body structure, it will be appreciated that microfluidic devices in general and the present invention in particular can take a variety of forms, including aggregations of various fluidic components such as capillary tubes, individual chambers, arrangement of channel(s) etc., that are pieced together to provide the integrated elements of the complete device. For example, FIGS. 1-6, illustrate some of many possible arrangements of the elements of the present invention.

### Device Arrangement

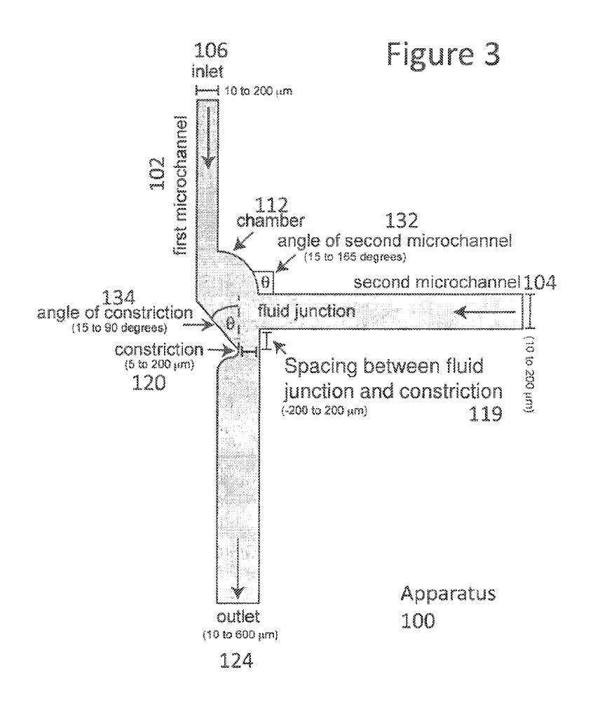

In one such possible arrangement, as shown in FIG. 1, a microfluidic apparatus 100 for merging or diluting droplets is shown. The microfluidic apparatus has a first microchannel 102 and a second microchannel 104. The first microchannel comprises an inlet portion 106 fluidly connected to a chamber portion 112 fluidly connected to an outlet portion 124. The inlet portion 106 transfers fluid comprising droplets along a first flow axis 110 into a chamber portion 112. The inlet portion 106 has an inlet portion cross-sectional area 110, defined as the product of the width and height of the inlet portion, where the width and height are orthogonal to the first flow axis. The chamber portion comprises a second flow axis 114, a maximal cross-sectional area, a fluid junction 116 having a fluid junction cross-sectional area 118, a constriction 120 having a constriction cross-sectional area 122, and a spacing 119 between the fluid junction and constriction. The maximal cross-sectional area is defined as the maximum of the product of the width and the height in the chamber portion, where the width and height are orthogonal to the second flow axis. The second flow axis is the average flow angle of fluid in the fluid junction as it travels from the inlet portion 106 and second microchannel 104, through the chamber portion 112, through the constriction point 120, and into the outlet portion 124. The second flow axis is shaped in-part by the angle of constriction 134 (see FIG. 3) of the walls of the chamber portion which connect to the constriction. The constriction is formed at the minimum cross-sectional area orthogonal to the direction of flow through the chamber portion and into the outlet portion. Thus, the constriction cross-sectional area is defined as the product of the width and height orthogonal to the flow axis in the constriction. The outlet portion has an outlet portion cross-sectional area 126 and a third flow axis 128. The outlet portion cross-sectional area 126 is the product of the width and height of the outlet portion, where the width and height are orthogonal to the third flow axis. The second microchannel 104 is fluidly connected to the first microchannel 102 at the fluid junction 116. The second microchannel transfers fluid along a fourth flow axis 130 into the chamber portion 112 through the fluid junction 116. The angle 132 between the first flow axis and fourth flow axis is represented in FIG. 1 by “Θ.”

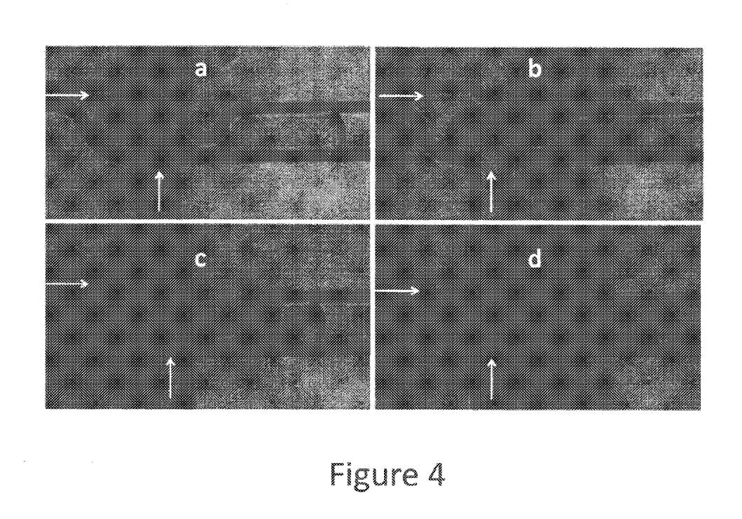

The chamber portion 112 facilitates the merger of a droplet from the inlet portion 106 and fluid from the second microchannel 104. The fluid from the second microchannel and the fluid in the droplet are miscible. In one embodiment, the droplet fluid is a non-polar solution, i.e., fat or oil. In another embodiment, the droplet fluid is a polar solution, i.e., water or methanol. The fluid from the second microchannel could be in the form of a droplet. In a further embodiment, the droplet is an aqueous-in-oil microdroplet, which can be used to perform a variety of biochemical and molecular biological tasks. In one embodiment shown in FIG. 4, panels a-d, the chamber portion tapers or narrows along the flow axis, merging the droplet from the inlet portion and the fluid from the second microchannel In FIG. 4a, the droplet flows out of the inlet portion into the chamber portion in the direction of the white arrow, and a bud of fluid forms at the fluid junction of the second microchannel In FIG. 4b, the droplet merges with the bud. In FIG. 4c, the constriction downstream from the bud pinches off the merged droplet. In FIG. 4d, a diluted or merged droplet flows out of the constriction and into the outlet portion. The droplet merging as shown is useful, for example, to add reagents, biological materials, synthetic materials, or other components to a droplet or to dilute a droplet.

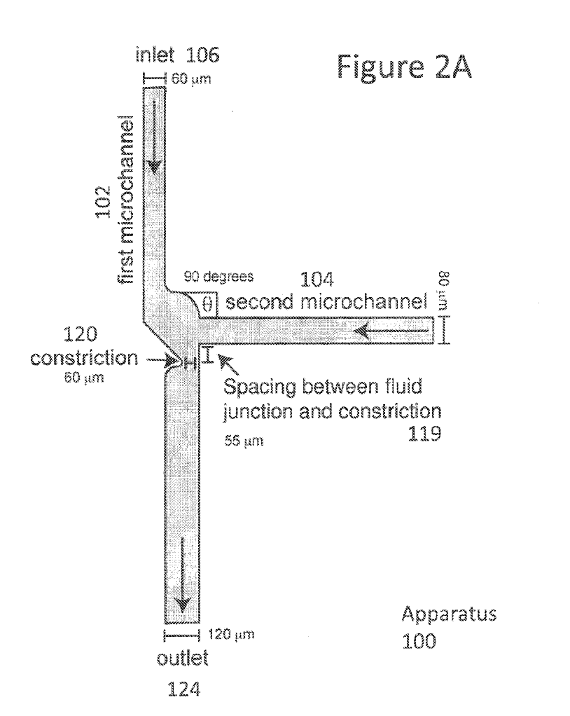

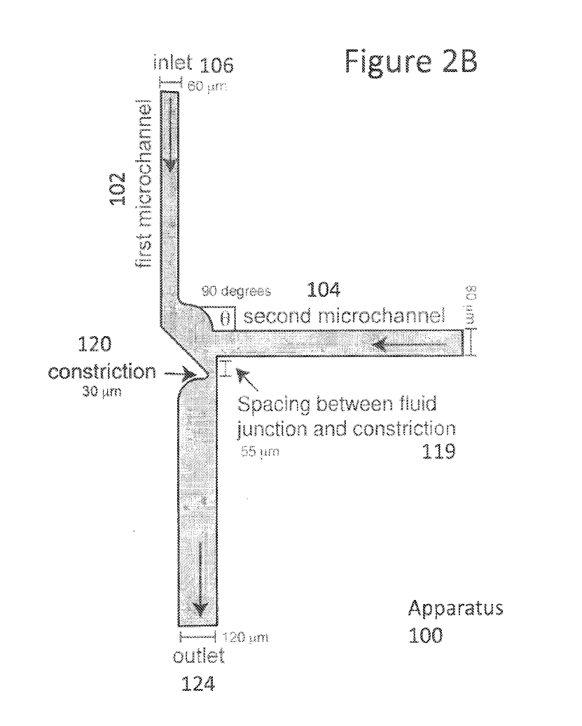

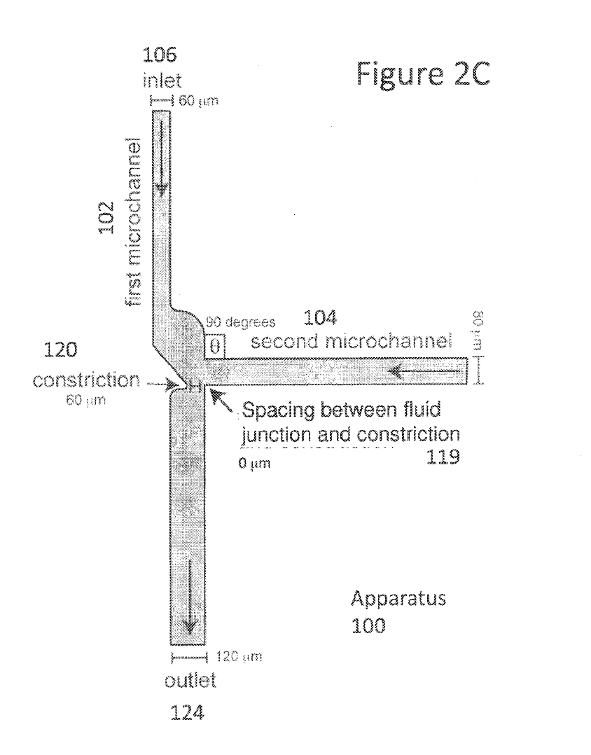

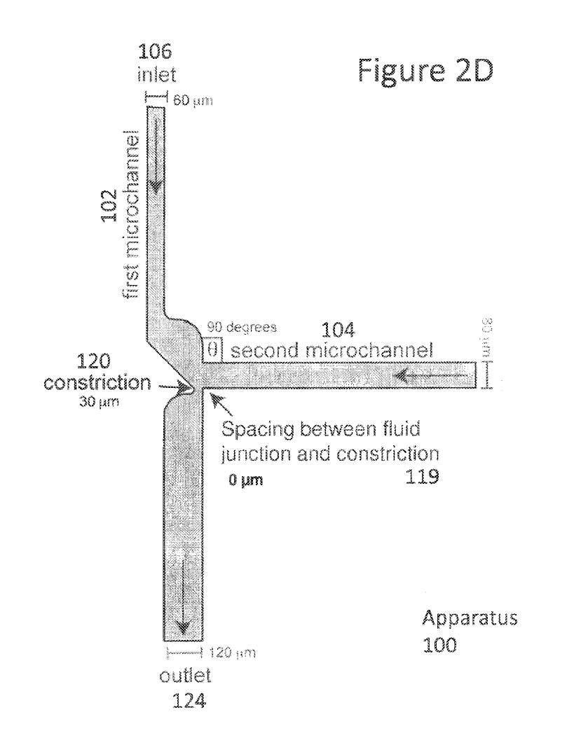

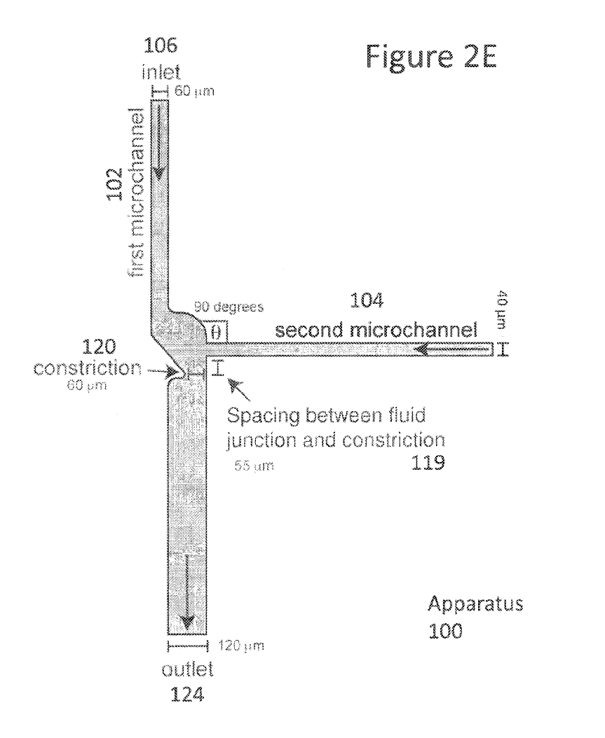

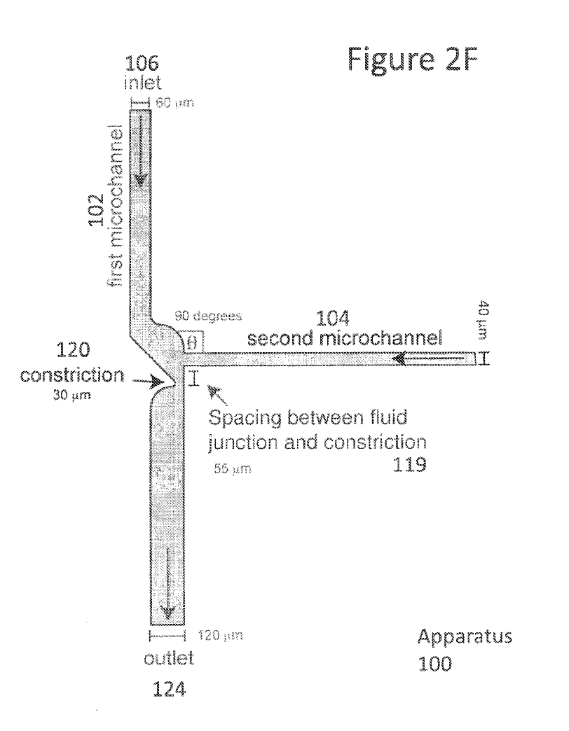

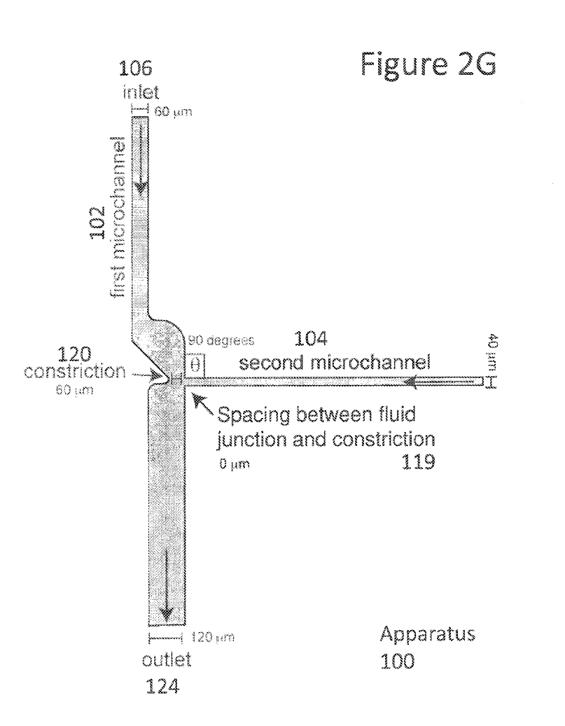

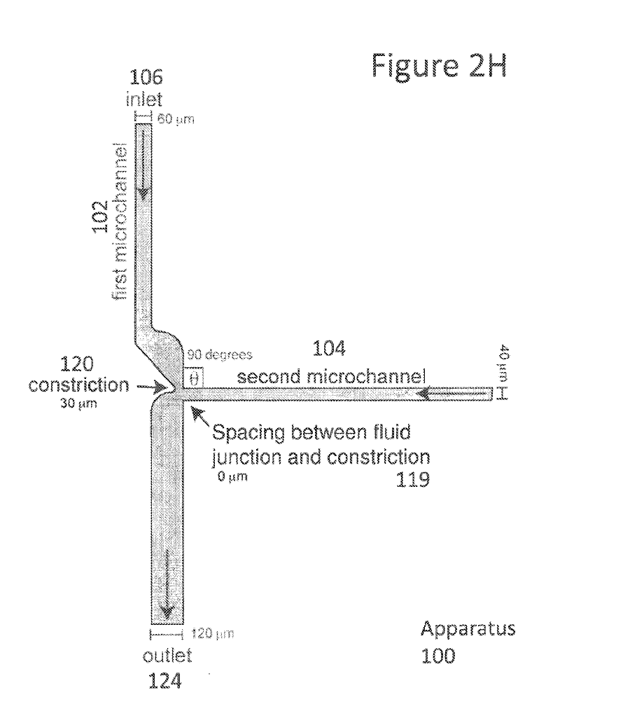

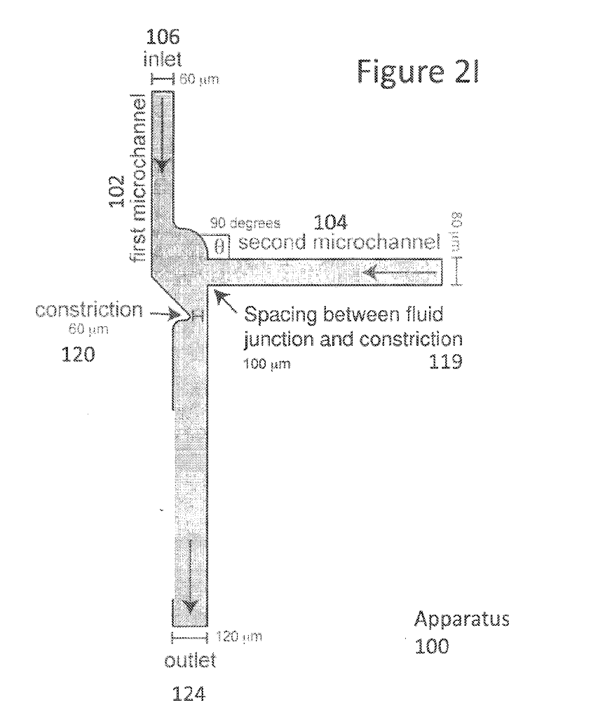

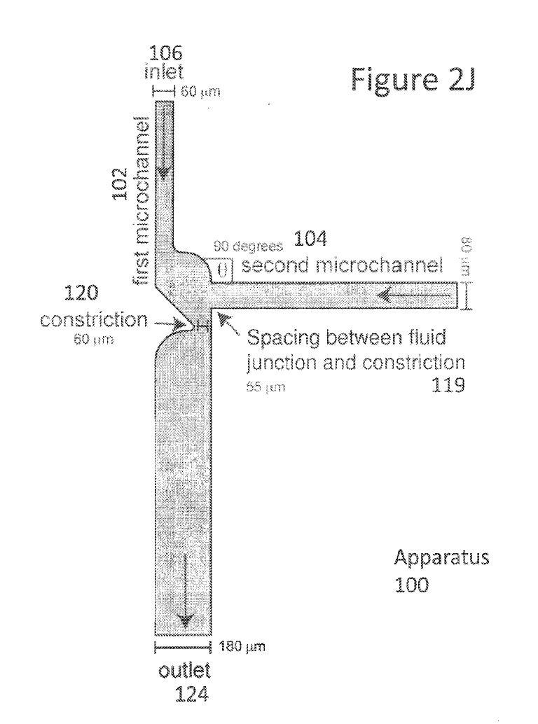

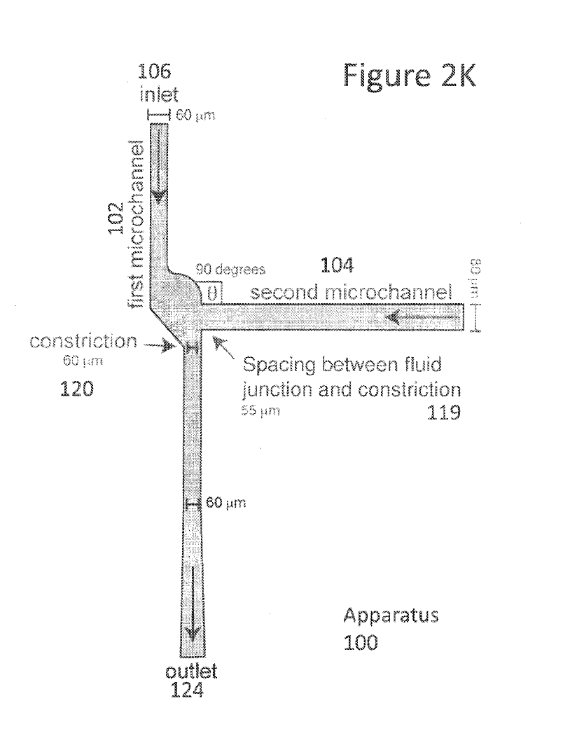

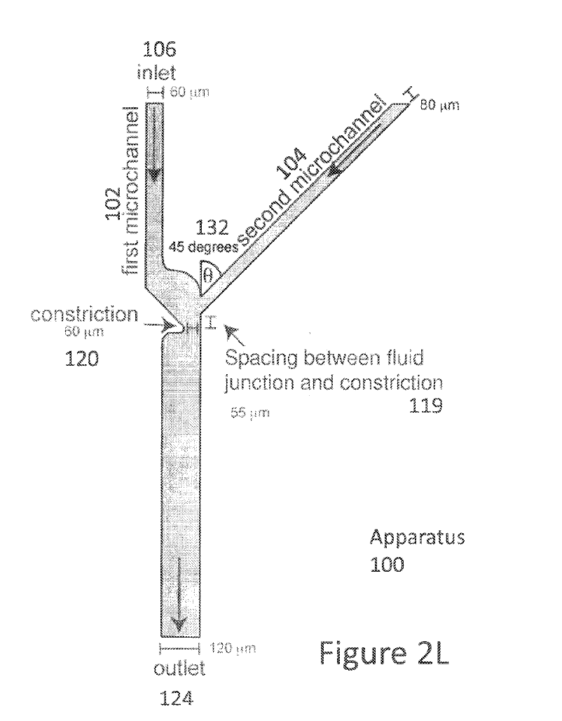

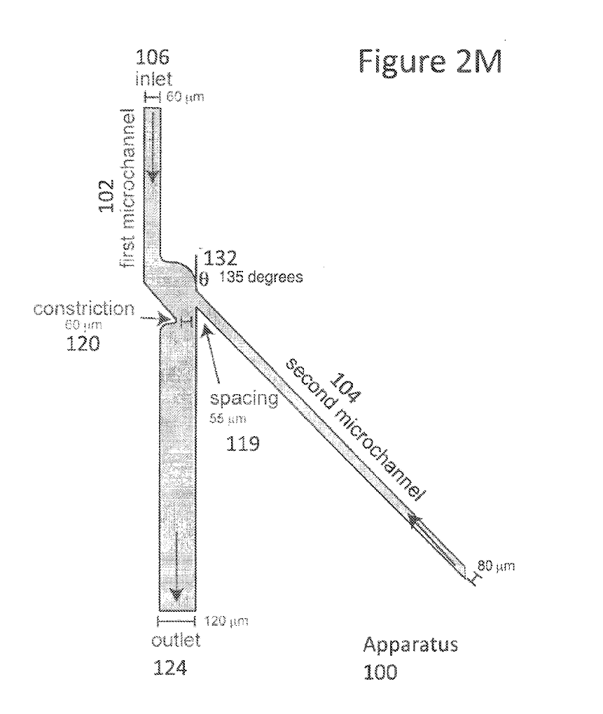

Selected embodiments and configurations of the microchannels are provided in FIG. 2, A-M. In one embodiment, the maximal width of the chamber portion is at least three times the width of the inlet portion. In another embodiment, the maximal width of the chamber portion is at least two times the width of the second microchannel In one embodiment shown in FIG. 2A, the inlet portion 106 has a width of 60 μm, the fluid junction 116 has a width of 80 μm, the constriction 120 has a width of 60 μm, and the outlet portion 124 has a width of 120 μm. The spacing 119 between the fluid junction and the constriction is 55 μm. The angle 132 between the inlet portion and the second microchannel is 90 degrees. In another embodiment as shown in FIG. 2B, the width of the constriction 120 is 30 μm. In still another embodiment shown in FIG. 2C, the fluid junction is directly adjacent to the constriction. In a further embodiment shown in FIG. 2D, the width of the constriction 120 is 30 μm and the fluid junction is directly adjacent to the constriction. In an alternative embodiment shown in FIG. 2E, the width of the fluid junction 116 is 40 μm. In a further embodiment shown in FIG. 2F, the width of the fluid junction is 40 μm, and the constriction 120 is 30 μm. In one aspect shown in FIG. 2G, the width of the fluid junction is 40 μm, and the fluid junction is directly adjacent to the constriction. In a further aspect shown in FIG. 2H, the width of the fluid junction 116 is 40 μm, the fluid junction is directly adjacent to the constriction, and the width of the constriction 120 is 30 μm. In another aspect shown in FIG. 21, the spacing 119 between the fluid junction and the constriction is 100 μm. In still another aspect as shown in FIG. 2J, the width of the outlet portion 124 of the first microchannel is 180 μm. In yet another aspect as shown in FIG. 2K, the width of the outlet portion 124 of the first microchannel and of the constriction is 60 μm. The outlet portion widens downstream from the constriction point. In one embodiment, shown in FIG. 2L, the angle 132 between the first flow axis 110 and the fourth flow axis 130 is 45 degrees. In another embodiment, shown in FIG. 2M, the angle 132 between the first flow axis 110 and the fourth flow axis 130 is 135 degrees. FIG. 2A-M provide some examples of different possible designs and measurements associated with components of the device. A variety of other sizes and shapes can be used for the components. Furthermore, the components of different sizes can be combined in a variety of ways, including other than the combinations shown in these figures.

Selected ranges of embodiments of the invention are provided in FIG. 3. In these aspects, the width of the inlet portion 106 is between 10 and 200 μm, the width of the constriction 120 is between 5 and 200 μm, the width of the outlet portion 124 is between 10 and 600 μm, and the width of the fluid junction interface 116 between the chamber portion 112 and the second microchannel 104 is between 10 to 200 μm (the possible sizes can also include the endpoints values of each of these ranges). The widths of each of these components can include any ranges within these ranges provided in FIG. 3 (i.e., the inlet portion could be between 10 and 100 μm or between 14 and 16 μm) or any values or fractional values within these ranges (i.e., the inlet portion could be 25 μm, 50 μm, 65.2 μm, and so forth). In these aspects, the angle of constriction 134 is between 15 and 90 degrees, while the angle 132 between the first flow axis 110 of the inlet portion 106 and the fourth flow axis 130 of the second microchannel 104 is between 15 and 165 degrees. These angles can include the endpoints of these ranges, ranges within these ranges, or any values or fractional values within these ranges. Also in these aspects, the space 119 between the fluid junction and the constriction is between −200 and 200 μm, where a distance below zero represents the distance of the fluid junction after the constriction along the flow axes. These constriction size can include the endpoints of this range, ranges within this ranges, or any values or fractional values within this range.

### Method for Droplet Merging

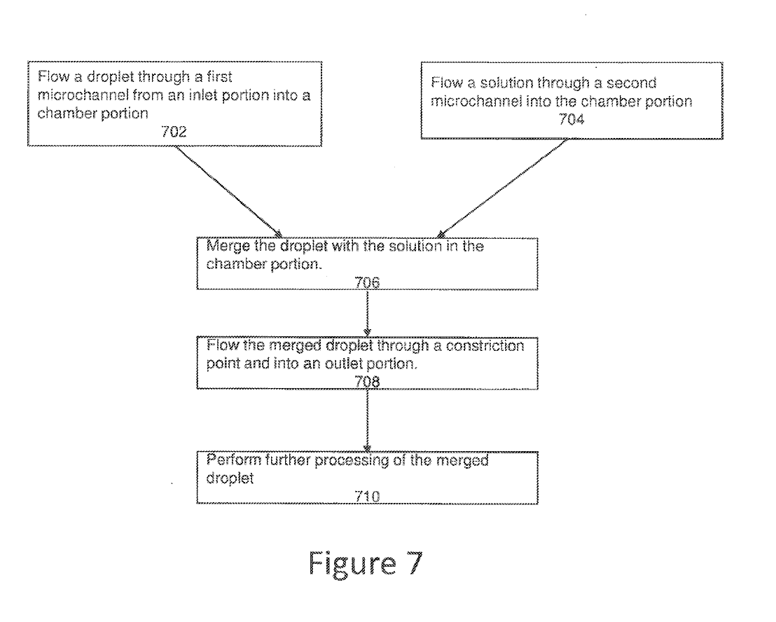

Selected embodiments of the microfluidic device described above are useful for merging a droplet with a miscible fluid. In one example, as shown in the flowchart in FIG. 7, a droplet is flowed 702 through a first microchannel from an inlet portion into a chamber portion. In parallel, a solution for merging with the droplet is flowed 704 through a second microchannel into the chamber portion. The droplet merges 706 with the solution in the chamber portion, and flows 708 through a constriction point and into an outlet portion. The walls of the chamber portion narrow in the direction of the second flow axis, and the droplet merges with the solution due to increased pressure pushing the solution and the droplet together. The merged droplet and solution flow through the constriction point, where the merged droplet is pinched off. Further processing 710 can be performed on the merged droplet. This method can be performed using the microfluidic devices presented in FIGS. 1-4, but can also be performed using other microfluidic devices having different designs or including only some of the components of the microfluidic devices presented in FIGS. 1-4.

The microfluidic devices of the present invention can include other features of microscale systems, such as fluid transport systems which direct droplet and fluid movement within the microchannels, incorporating any movement mechanism set forth herein (e.g., fluid pressure sources for modulating fluid pressure in the array channel, electrokinetic controllers for modulating voltage or current in the array channel, gravity flow modulators, magnetic control elements for modulating a magnetic field within the array channel, or combinations thereof). For example, droplet flow is optionally carried out in a pressure-based system

Pressure is optionally applied to microscale elements, e.g., to a channel, region, or chamber, to achieve fluid movement using any of a variety of techniques. Fluid flow and flow of materials suspended or solubilized within the fluid, including droplets, cells, or particle sets, is optionally regulated by pressure based mechanisms such as those based upon electrokinetic or electroosmotic fluid flow, fluid displacement, e.g., using a piston, pressure diaphragm, vacuum pump, probe or the like to displace liquid and raise or lower the pressure at a site in the microfluidic system. The pressure is optionally pneumatic, e.g., a pressurized gas, or uses hydraulic forces, e.g., pressurized liquid, or alternatively, uses a positive displacement mechanism, i.e., a plunger fitted into a material reservoir, for forcing material through a channel or other conduit, or is a combination of such forces. In one embodiment, the fluid pressure sources provide sufficient pressure to merge 10 droplets per second. In another embodiment, more than 5, 10, 20, or 20 droplets are merged per second.

In some embodiments, a vacuum source is applied to a reservoir or well at one end of a channel to draw a fluidic material through the channel For example, a vacuum source is optionally situated for drawing fluid into or through a microchannel, e.g., for drawing a droplet into a chamber portion. Pressure or vacuum sources are optionally supplied external to the device or system, e.g., external vacuum or pressure pumps sealably fitted to the inlet or outlet of the channel, or they are internal to the device, e.g., microfabricated pumps integrated into the device and operably linked to the channel.

Hydrostatic, wicking and capillary forces are also optionally used to provide fluid pressure for continuous fluid flow of materials such as enzymes, substrates, modulators, or protein mixtures. In these methods, an adsorbent material or branched capillary structure is placed in fluidic contact with a region where pressure is applied, thereby causing fluid to move towards the adsorbent material or branched capillary structure. The capillary forces are optionally used in conjunction with pressure-based flow in the present invention. The capillary action pulls material through a channel For example, a wick is optionally added to draw fluid through a porous matrix fixed in a microchannel or chamber.

### Methods for Cell Lysis

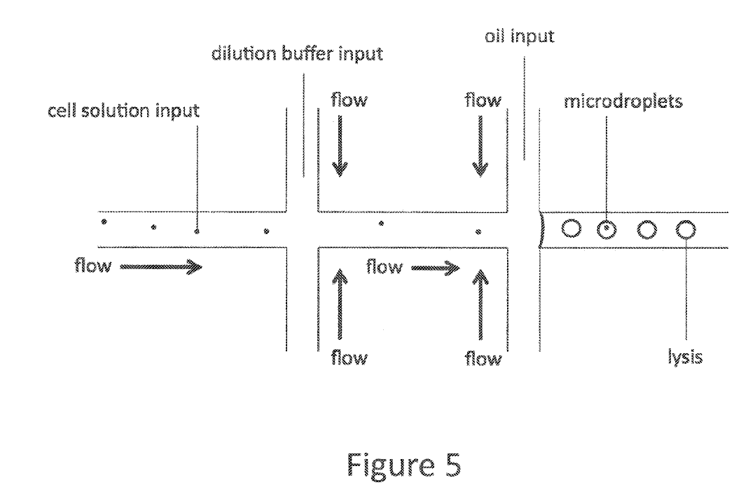

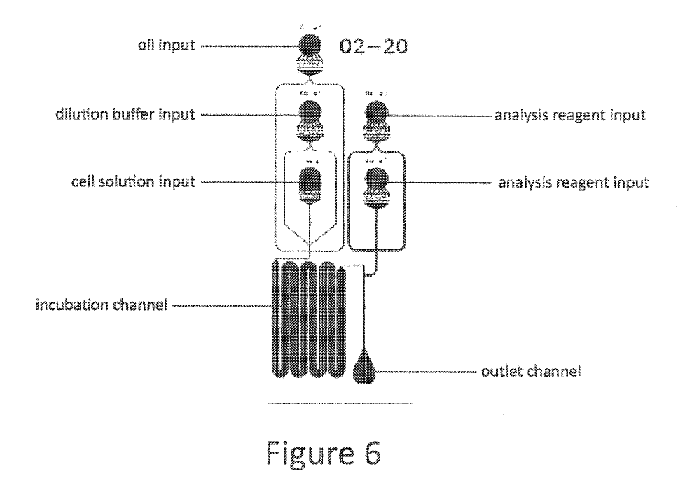

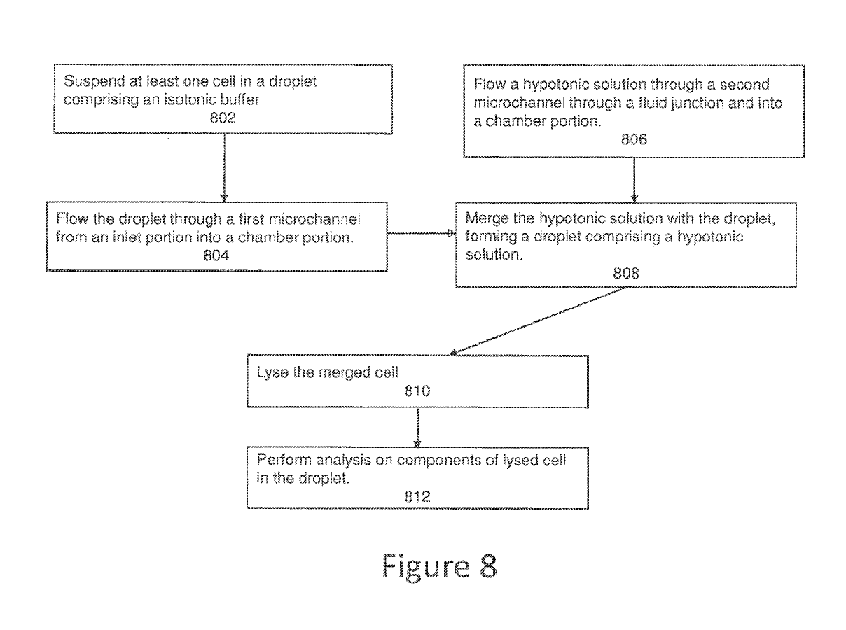

In some embodiments of the invention, methods for cell lysis are provided. One aspect of a microfluidic schematic for lysing cells in microdroplets is provided in FIG. 5, though the microfluidic devices shown in FIGS. 1-4 can also be used. In one embodiment of the method, as shown in FIG. 8, at least one cell is suspended 802 in a droplet comprising an isotonic solution, and is flowed 804 through a first microchannel from an inlet portion into a chamber portion (shown in FIG. 5 as the cell solution input). In parallel, a solution comprising a hypotonic solution (as compared to the cell), i.e., a dilution buffer input in FIG. 5, is flowed 806 through a second microchannel through a fluid junction and into a chamber portion. In FIG. 5, the chamber portion can include any portion of the microchannel in which the cell solution input and dilution buffer input meet, and this chamber can be the same size as one or both of the first and second microchannels, as shown in FIG. 5, or differently sized from one or both of the first and second microchannels, as shown in FIGS. 1-4. In the chamber portion, the hypotonic solution merges 808 with the droplet comprising the cell. For some designs of the chamber, this is due to the increased pressure formed by the tapering walls of the chamber portion leading to a constriction (e.g., see FIGS. 1-4). The merger of the droplet with the hypotonic solution produces a merged droplet comprising a cell and a hypotonic solution. The hypotonic solution within the droplet lyses 810 the cell, while the droplet is maintained. This embodiment of the method results in the components of a lysed cell captured in a droplet in a microfluidic channel The embodiment of FIG. 5 shows that there can also be an oil input that results in the formation of microdroplets in which the lysis occurs. After lysis, analysis 812 of components of the lysed cell contained in the droplet can be performed. The method described here is useful, for example, for biochemical analysis of cellular components, i.e., nucleic acid analysis. The invention is useful in a number of biological and medical fields, including immunology, prenatal diagnosis, and cancer diagnosis. One embodiment of a microfluidic design for achieving the methods of cell lysis of the present disclosure is shown in FIG. 6.

In another embodiment, cells are suspended in an isotonic buffer comprising a salt, protein, or other chemicals to stabilize the cell. The cells are encapsulated by droplets comprising the isotonic buffer in oil containing less than a microliter of buffer. The droplets are simultaneously diluted with a second stream of hypotonic buffer with at least one order of magnitude lower concentration of salt, protein, or other chemicals than the isotonic buffer during cell encapsulation.

In one aspect, the hypotonic solution is a dilution buffer. In another aspect, the hypotonic solution is highly compatible with subsequent biochemical analysis of cellular components. In still another aspect, the hypotonic solution comprises reagents, biological materials, or synthetic materials for biochemical analysis. In some aspects, heat inactivation or purification of the cellular components for subsequent biochemical analysis are not necessary. For example, direct addition to the lysed cell and lysis buffer mix of the components for polymerase chain reaction (PCR) can be used to amplify and detect polynucleic acid sequences. In the context of high-throughput methods for cellular analysis (e.g., 100 Hz), this is particularly useful, because additional steps such as washing and heating require more complex microfluidic instrumentation.

In another embodiment, chemical lysis is used in combination with, or instead of, hypotonic lysis. In this embodiment, cells are suspended in an isotonic buffer comprising a salt, protein, or other chemicals to stabilize the cell. The cells are encapsulated by droplets comprising the isotonic buffer in oil, each droplet containing less than a microliter of isotonic buffer. The droplets are merged with a second solution of lysis buffer. The lysis buffer effects lysis of any cellular material in the droplets. This lysis will occur, for example immediately at room temperature, or in certain embodiments after heating to 90-98° C.

In one aspect, the chemical solution is a lysis buffer. In another aspect, the lysis solution is highly compatible with subsequent biochemical analysis of cellular components. In still another aspect, the hypotonic solution comprises reagents, biological materials, or synthetic materials for biochemical analysis. In some aspects, heat inactivation or purification of the cellular components for subsequent biochemical analysis are not necessary. For example, direct addition to the lysed cell and lysis buffer mix of the components for polymerase chain reaction (PCR) can be used to amplify and detect polynucleotide sequences. In the context of high-throughput methods for cellular analysis (e.g., 100 Hz), this is particularly useful, because additional steps such as washing and heating require more complex microfluidic instrumentation.

The present disclosure has broad applicability in many areas of biological analysis. The useful application can include the fields of cancer diagnostics, immunology, or infectious disease diagnostics. For example, vertebrate immune systems are comprised of millions of different types of immune cells. Embodiments of the invention can be used to lyse and analyze millions of immune cells in parallel. The methods provided could be used on many different kinds of cells, from lower-complexity cells such as bacteria to higher-complexity cells such as mammalian cells. The methods provided could also be used to analyze heterogeneous or homogeneous subpopulations of cells, such as subpopulations of tumors.

In the preceding detailed description, reference has been made to the accompanying drawings that form a part hereof, and in which are shown by way of illustration specific embodiments in which the present invention may be practiced. These embodiments, and certain variants thereof, have been described in sufficient detail to enable those skilled in the art to practice embodiments of the present invention. It is to be understood that other suitable embodiments may be utilized and that logical, mechanical, chemical and electrical changes may be made without departing from the spirit or scope of such inventive disclosures. To avoid unnecessary detail, the description omits certain information known to those skilled in the art. The preceding detailed description is, therefore, not intended to be limited to the specific forms set forth herein, but on the contrary, it is intended to cover such alternatives, modifications, and equivalents, as can be reasonably included within the spirit and scope of corresponding claims.

It should be noted that the language used herein has been principally selected for readability and instructional purposes, and it cannot have been selected to delineate or circumscribe the inventive subject matter. Accordingly, the disclosure is intended to be illustrative, but not limiting, of the scope of claimed methods.

Any terms not directly defined herein shall be understood to have the meanings commonly associated with them as understood within the art of the present disclosure. Certain terms are discussed herein to provide additional guidance to the practitioner in describing the compositions, devices, methods and the like of aspects of the present disclosure, and how to make or use them. It will be appreciated that the same thing can be said in more than one way. Consequently, alternative language and synonyms can be used for any one or more of the terms discussed herein. No significance is to be placed upon whether or not a term is elaborated or discussed herein. Some synonyms or substitutable methods, materials and the like are provided. Recital of one or a few synonyms or equivalents does not exclude use of other synonyms or equivalents, unless it is explicitly stated. Use of examples, including examples of terms, is for illustrative purposes only and does not limit the scope and meaning of the aspects of the present disclosure herein.

1. A method for increasing the volume of a droplet, comprising:
flowing a first solution comprising an initial droplet in an immiscible carrier liquid through a first microchannel from an inlet portion defining a first fluid path into a chamber portion, said chamber portion comprising a constriction downstream from said inlet portion;
flowing a second solution from a second microchannel through a fluid junction to said chamber portion, wherein said second solution is miscible with said initial droplet, and said initial droplet and said second solution merge within said chamber portion, thereby increasing the volume of said initial droplet and forming a merged droplet.

2. The method of claim 1, wherein said merged droplet flows through the constriction and wherein the flow of said first and second solutions through said constriction produces a pressure gradient within said chamber portion.

3. The method of claim 1, wherein said initial droplet diameter is less than 300 μm.

4. The method of claim 1, wherein a rate of formation of said merged droplets is greater than one Hz.

5. The method of claim 4, wherein the rate of formation of said merged droplets is equal or greater than a rate selected from 1, 5, 10, 20, 50, 75, and 100 Hz.

6. The method of claim 1, wherein said first solution or said second solution flows under pressure generated by a pump.

7. The method of claim 1, wherein said first solution and said second solution are polar.

8. The method of claim 1, wherein said first solution and said second solution are non-polar.

9. The method of claim 1, wherein said second solution comprises at least one of a reagent, a biological material, and a synthetic material.

10. The method of claim 1, wherein said second solution dilutes said initial droplet.

11. The method of claim 1, wherein said initial droplet comprises a cell.

12. The method of claim 11, wherein said second solution is hypotonic as compared to a tonicity of said initial droplet.

13. The method of claim 12, wherein said hypotonicity of said second solution promotes lysis of said cell.

14. The method of claim 11, wherein the second solution comprises a chemical that promotes lysis of the cell.

15. The method of claim 14, wherein said chemical is a surfactant.

16. The method of claim 15, wherein said surfactant is selected from the group consisting of:
Triton X-100, Tween 20, and NP 40.

17. The method of claim 14, wherein said chemical is an enzyme.

18. The method of claim 17, wherein said enzyme is proteinase K.

19. The method of claim 14, wherein said lysis of the cell occurs at room temperature.

20. The method of claim 14, wherein said lysis of the cell occurs at 90-98° C.

21. A method for cell lysis, comprising:
flowing a first solution comprising a cell encapsulated in an initial droplet in an immiscible carrier liquid through a first microchannel defining a first fluid path from an inlet portion into a chamber portion, said chamber portion comprising a constriction downstream from said inlet portion;
flowing a second solution from a second microchannel through a fluid junction to said chamber portion, wherein said second solution comprises a cell lysis solution miscible with said initial isotonic droplet, and said initial isotonic droplet and said second solution merge within said chamber portion, thereby diluting the initial isotonic droplet with said second solution, creating a merged droplet, and wherein said cell encapsulated in said merged droplet lyses.

22. A microfluidic device, comprising:
a first microchannel defining a first fluid path, said microchannel comprising:
an inlet portion comprising an inlet portion cross-sectional area and a first flow axis;
a chamber portion in fluid communication with and adjacent to said inlet portion, comprising a maximal cross-sectional area and a second flow axis, the chamber portion comprising a fluid junction comprising a fluid junction cross-sectional area and a constriction comprising a constriction cross-sectional area, wherein said constriction cross-sectional area is less than the maximal cross-sectional area of the chamber portion; and
an outlet portion in fluid communication with and adjacent to said chamber portion, comprising an outlet portion cross-sectional area and a third flow axis, wherein said outlet portion cross-sectional area is greater than or equal to said constriction cross-sectional area; and
a second microchannel defining a second fluid path and a fourth flow axis, said second fluid path terminating at one end at said fluid junction.
